(19) United States
(12) Patent Application Publication
(10) Pub. No.: US 2020/0258219 A1
(43) Pub. Date: Aug. 13, 2020

(54) METHODS FOR TRAINING A BREAST CANCER SCREENING MODEL VIA THERMOGRAPHIC IMAGE PROCESSING AND THERMAL BREAST SIMULATION

(71) Applicant: Higia, Inc., Wilmington New Castle, DE (US)

(72) Inventors: Julián Ríos Cantú, Monterrey (MX); Francisco Javier González, San Luis Potosí (MX); Raymundo González Leal, Monterrey (MX); Juan Carlos Pendas, Peubla (MX)

(21) Appl. No.: 16/744,046

(22) Filed: Jan. 15, 2020

Related U.S. Application Data
(60) Provisional application No. 62/792,894, filed on Jan. 15, 2019.

(51) Int. Cl. G06T 7/00; A61B 5/00; A61B 5/01; G06T 7/187

(52) U.S. Cl. CPC G06T 7/0012; A61B 5/6823; A61B 5/015; A61B 5/4312; A61B 2562/0271; G06T 2207/30068; G06T 2207/30096; G06T 2207/20081; G06T 2207/20084; G06T 7/187

(57) ABSTRACT

A method for training a breast cancer screening model includes: accessing a set of thermographic images of a torso of a patient, the set of thermographic images associated with a pathology label. The method also includes, for each of the set of thermographic images: calculating an image mask defining a breast area; within the image mask, distributing a set of sampling points, the set of sampling points approximating locations of temperature sensors in a sensor mesh of a temperature sensing brassiere if the temperature sensing brassiere were worn over the torso of the patient; for each sampling point in the set of sampling points, extracting a temperature data point from the set of thermographic images to generate a temperature map; generating an input vector based on the temperature map; and inputting the input vector and the pathology label as a training example in a breast cancer screening model.

### CROSS REFERENCE TO RELATED APPLICATIONS

This Application claims the benefit of U.S. Provisional Application No. 62/792,894, filed on 15 Jan. 2019, which is incorporated in its entirety by this reference.

This Application is related to U.S. patent application Ser. No. 16/534,993, filed on 7 Aug. 2019, which is incorporated in its entirety by this reference.

### TECHNICAL FIELD

This invention relates generally to the field of medical diagnostics and more specifically to a new and useful method for breast health monitoring in the field of medical diagnostics.

### BRIEF DESCRIPTION OF THE FIGURES

FIG. 1 is a flowchart representation of a first method;

FIG. 2 is a flowchart representation of a second method;

FIG. 3 is a schematic representation of a temperature-sensing brassiere; and

FIGS. 4A, 4B, and 4C is a schematic representation of the temperature-sensing brassiere.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. First Method

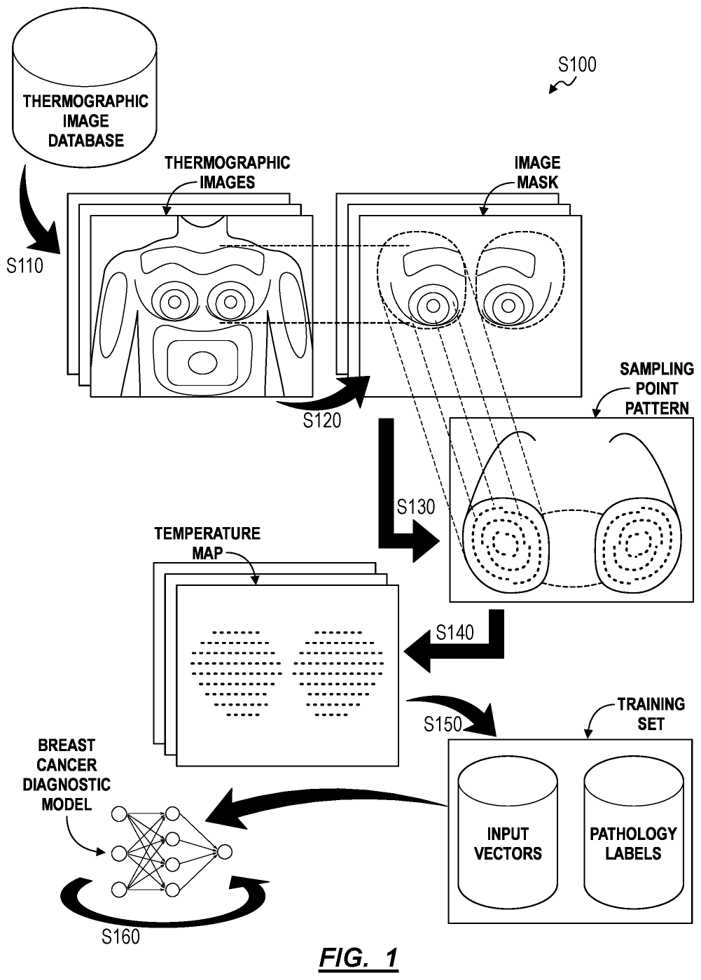

As shown in FIG. 1, a method S100 for training a breast cancer screening model includes: accessing a set of thermographic images of a torso of a patient, the set of thermographic images associated with a pathology label in Block S110; for each of the set of thermographic images, calculating an image mask defining a breast area in the thermographic image in Block S120; within the image mask, distributing a set of sampling points of each thermographic image, the set of sampling points approximating locations of temperature sensors in a sensor mesh of a temperature sensing brassiere if the temperature sensing brassiere were worn over the torso of the patient in Block S130; for each sampling point in the set of sampling points, extracting a temperature data point from the set of thermographic images to generate a temperature map in Block S140; generating an input vector based on the temperature map in Block S150; and inputting the input vector and the pathology label as a training example in a breast cancer screening model in Block S160.

### 1.1 First Method: Applications

Generally, the first method S100 is executed by a computer system (hereinafter “the system”), which can include a set of computational devices connected over a network (e.g., the internet) and a local computing device configured to display an interface to a user of the system. The system executes the first method S100 in order to leverage preexisting thermographic image data, obtained via thermographic breast cancer screenings, to train a breast cancer screening model for a contact thermography device (e.g., a temperature-sensing brassiere). The temperature-sensing brassiere includes a sensor mesh and a set of discrete temperature sensors (e.g., 196 temperature sensors) spread across the surface of the patient's breasts by the sensor mesh. The temperature-sensing brassiere records temperature data from each of the discrete temperature sensors in the sensor mesh while worn by a patient. The system (or another computing device) can subsequently train and/or evaluate a breast cancer screening model—configured to output risk assessments or breast cancer pathology classifications for a population of patients—based on these temperature data.

Accuracy of the breast cancer screening model may depend on the quantity and quality of training data, which may take years to collect via tests administered with the temperature-sensing brassiere. However, the system can reduce the duration of this training period and increase the quantity of training data for the breast cancer screening model by: extracting temperature values from an existing thermographic image of a patient with a known cancer pathology at pixel locations in the thermographic image that correspond to positions of the discrete temperature sensors in the temperature-sensing brassiere (if worn by the patient) to form a temperature map; and repeating this process for each thermographic image in a large corpus of thermographic images in order to generate corpus of temperature maps. The system can then train the breast cancer screening model according to corpus of temperature maps and corresponding cancer pathologies. Thus, the method S100 can transform thermographic images into an input vector that can function as a training example for the breast cancer screening model.

More specifically, each temperature map contains mapping information that indicates a correspondence between a temperature data point extracted from the thermographic image and a particular sensor location of the temperature-sensing brassiere. For example, for a temperature-sensing brassiere including 196 temperature sensors the system would generate temperature maps with 196 temperature data points each corresponding to one of the sensors. Thus, each temperature map conveys spatial information in addition to temperature data based on the relative positions of the temperature sensors in the temperature-sensing brassiere.

The system executes the first method S100 to transform the thermographic images of a patient into a set of discrete temperature data points at approximate positions of temperature sensors of the temperature-sensing brassiere across the surface of the patient's breasts, if the temperature-sensing brassiere were worn over the patient's breasts. More specifically, the system generates a temperature map by: calculating an image mask of a breast area of a patient in a thermographic image; distributing a set of sampling points (in a sampling point pattern) over the image mask approximating the location of temperature sensors in a temperature-sensing brassiere worn over the patient's breasts; extracting a temperature data point for each sampling point in the thermographic image to form a temperature map; generating an input vector for a machine learning model based on the temperature map; and input the input vector and a pathology label as a training example for the machine learning model.

The system has access to a thermographic image database from which to retrieve a set of thermographic images depicting the upper torso of a patient (e.g., a female patient). The system can then access each image in order to sample temperature data from the image. In one implementation, the system can access lateral thermographic images and frontal thermographic images of the same patient in order to provide additional thermal data and breast shape information when sampling temperature data from the thermographic images.

Upon retrieving an image, the system can implement image segmentation techniques to detect a breast area in the image (i.e. a contiguous area of the image representing the temperature of the breast tissue of the patient), such as active contouring, morphological image segmentation, Hough transform segmentation, edge-based segmentation, curvature-based segmentation, or any other computer vision technique to identify the breast area of in the image. Additionally or alternatively, the system can receive an input from a user indicating a breast area in a thermographic image. Once the system has detected a breast area in the thermographic image, the system can define an image mask over the breast area in the image.

After defining the image mask, the system can distribute, over the image mask, a set of sampling points that approximate the locations of temperature sensors in a temperature-sensing brassiere worn over the patient's breasts. In order to distribute the sampling points in a pattern that approximates the distribution of temperature sensors of a temperature-sensing brassiere worn over the patient's breasts, the system can detect the location of the patient's areolas within the image mask and position the distribution of sampling points based on this location. Furthermore, the system can sample from an offset distribution (e.g., a normal distribution) in order to randomly offset each sampling point relative to the original distribution in order to represent random variation in the relative position of temperature sensors in the temperature-sensing brassiere.

Once the sampling points have been calculated, the system can extract temperature data from the thermographic image according to the color of the thermographic image at each sampling point (e.g., at a pixel location defined by each sampling point). Upon extracting the temperature data (e.g., as a temperature map) from the thermographic image, the system can store the extracted temperature data as an input vector for a breast cancer screening model. Additionally, the system can also perform further processing, such as data normalization, data interpolation, and/or data comparison between temperature data derived from each of the patient's breasts to generate the input vector for the breast cancer screening model.

The system can then pair each input vector with a corresponding pathology label describing the pathology of the patient. The system can then include the input vector and the pathology label in a supervised learning algorithm to train the breast cancer screening model.

For ease of explanation, the method S100 is described in the context of a temperature-sensing brassiere. However, the method S100 can be adapted for any other contact thermography device by modifying the sampling point pattern that corresponds to the sensor mesh of the temperature-sensing brassiere to a sampling point pattern that corresponds to an array of temperature sensors as configured on the contact thermography device. The system can also include other properties of the contact thermography device (e.g., the general structure, materials, and/or other physical properties of the device) in approximating the sampling point pattern for the thermographic image.

### 1.2 Thermographic Image Access

In Block S110, the system can access a thermographic image database to retrieve a set of thermographic images of patients such that the system can convert the thermographic images into a temperature map in order to train the breast cancer screening model according to Blocks of the first method S100. In one implementation, the system can retrieve a set of thermographic images for a single patient including a frontal thermographic image and two lateral thermographic images (e.g., a right and a left thermographic image). In one implementation, the system can also access a color-temperature correspondence key for the thermographic images in order to determine the correspondence of colors/hues of pixels in the thermographic images with surface temperatures of the patient's breasts.

### 1.3 Pathology Label

The system can access a pathology label for each patient associated with the thermographic images of the patient, such that the computational model can perform a supervised learning algorithm by comparing the risk assessment or pathology classification output by the breast cancer screening model for an image with the pathology label of the same image. The pathology label can indicate the presence or absence of cancerous masses (e.g., a binary label). Alternatively, the pathology label can indicate a breast image reporting and data system category (hereinafter “BI-RADS category”) between zero and six. In yet another alternative, the pathology label can indicate a breast cancer stage according to the breast cancer staging system (i.e. the TNM system overseen by the American Joint Committee on Cancer).

However, the pathology label can include any variable that categorizes breast cancer pathology or any benign mass pathology that may affect the thermal properties of the patient's breasts.

### 1.4 Image Mask

Once the system has accessed a set of thermographic images of a patient's breast, the system can calculate and/or define an image mask for each of the set of thermographic images in Block S120. The image mask (approximately) defines regions of each thermographic image that include a patient's breast surface tissue and/or tissue that would be covered by the temperature-sensing brassiere if the temperature-sensing brassiere were worn by the patient depicted in the thermographic image.

In one implementation, in order to calculate an image mask for each thermographic image, the system can perform any common image segmentation technique or combination of image segmentation techniques such as thresholding, K-means clustering, active contouring, morphological image segmentation, Hough transform segmentation, edge-based segmentation, curvature-based segmentation, region-growing image segmentation. Additionally, the system can perform a combination of image segmentation techniques such as thresholding followed by contour detection.

In another implementation, the system can provide an interface to a user (e.g., a health professional or individual with knowledge of breast anatomy) that enables the user to select areas of the image depicting the patient's breast tissues. The user can then determine the image mask for each of the thermographic images manually. In yet another implementation, the system can execute a hybrid approach of both computer vision techniques and manual selection. For example, the system can implement thresholding and edge detection techniques to identify the lower boundaries of the patient's breasts before providing an interface for a user to define the upper boundaries of the patient's breasts.

However, the system can calculate an image mask for a thermographic image defining a region depicting the patient's breasts in the thermographic image in any other way.

1.5 Anatomic Feature Detection

After the system defines an image mask for the thermographic image, the system can detect various anatomic features (e.g., the areolas of the patient or the axillary regions of the patient) of the patient in order to position and/or orient the sampling point pattern, further described below, within the image mask in order to best approximate the locations of temperature sensors of the temperature-sensing brassiere if the temperature-sensing brassiere were worn by the patient. The shape and size of a patient's torso necessarily effects the fit of the temperature-sensing brassiere over the patient's breasts and therefore also effects the exact position of the temperature sensors of the temperature-sensing brassiere over the patient's breasts. Thus, by detecting various anatomic features of the patient, the system can subsequently distribute sampling points across the image mask for a patient's breasts that more closely match actual locations of the temperature sensors of the temperature-sensing brassiere if the temperature-sensing brassiere were worn by the patient.

In one implementation, the system can implement similar techniques to those described above with reference to defining the image mask in order to detect the areolas of the patient within the image mask. The system can detect the center or centroid of the patient's areolas as a pixel location within the image mask. Additionally or alternatively, the system can implement computer vision techniques to identify the lateral borders of the patient's axillary region and/or torso and thereby estimate the torso width of the patient. Furthermore, the system can measure a protrusion dimension of the patient's breasts (e.g., the distance from the frontal plane of the patient's body to the greatest extent of the patient's breasts), in a lateral thermographic image of the patient's breasts.

The system can also provide an interface that enables a user to define the location, orientation, and/or dimension of any of the aforementioned anatomical features. Furthermore, the system can implement a hybrid process that combines computer vision techniques and manual input from a user in order to obtain more accurate identification of anatomical features of the patient.

However, the system can identify anatomic features of a patient in an image mask of a thermographic image in any other way.

### 1.6 Sampling Point Pattern

Once the system defines an image mask for the region of the thermographic image depicting the patient's breasts, the system can distribute a set of sampling points within the image mask of each thermographic image, wherein the set of sampling points approximates locations of temperature sensors in a sensor mesh of a temperature sensing brassiere if the temperature sensing brassiere were worn over the torso of the patient in Block S130. More specifically, the system can select a set of sampling points (e.g., pixel locations within the image mask) that approximate, by two-dimensional projection, the location of each temperature sensor in the sensor mesh of the temperature-sensing brassiere by: estimating an approximate breast size and/or shape; applying a transformation to an initial distribution of sampling points approximating the deflection and conformation of the temperature sensors of the temperature-sensing brassiere if worn around the patient's breasts; shifting the initial distribution of sampling points based on sampling a fitting distribution that represents typical fitting variability of the temperature-sensing brassiere; and shifting each sampling point based on sampling a sensor position distribution that represents typical variability of the temperature sensors in the sensor mesh of the temperature-sensing brassiere.

The system can estimate the size and/or shape of the patient's breasts in order to determine the degree and manner with which the sensor mesh would be expected to stretch over the patient's breasts. In one implementation, the system can estimate the approximate chest circumference and cup size of the patient based on the image mask and/or any anatomical feature identified within the image mask. The system can then access a database of sampling point patterns approximating the temperature sensor distribution that corresponds to each common combination of chest circumference and cup size. Alternatively, the system can only access sampling point patterns corresponding to a breast cup size alone or a chest circumference alone.

In one implementation, the system can: generate a three-dimensional model of the patient's breasts based on frontal thermographic images, lateral thermographic images, and/or any identified anatomical features in the thermographic images; and simulate the elastic sensor mesh worn over the three-dimensional model. The system can then project the three-dimensional locations of each simulated temperature sensor in the simulated sensor mesh back on to the front and lateral thermographic images.

In another implementation, the system utilizes a standard sampling point pattern and scales the distribution based on the estimated torso size of the patient so that the standard sampling point pattern covers the entire image mask of the thermographic image.

In yet another implementation, the system places the sensors based only on the shape of the image mask and/or identified anatomical features of the breast (provided by a user via an interface or detected by the system). Thus, the system can generate a sampling point pattern for a thermographic image without simulating the breasts and/or torso of the patient directly.

After adapting the sampling point pattern to the particular patient, as represented in the set of thermographic images of the patient, the system can then: sample a fitting distribution to obtain a fit variability offset for the sampling point pattern; and shift each sampling point in the sampling point pattern by the fit variability offset. Thus, the system can shift the entire sampling point pattern by a randomly selected offset relative the image mask in order to account for variability in fit and placement of the temperature-sensing brassiere that may occur during a typical testing procedure. In one implementation, the system can sample fitting distribution multiple times in order to obtain multiple fit variability offsets to apply to different regions of the sampling point pattern. For example, the system can sample the fitting distribution twice and apply different fit variability offsets to the left and right side of the sampling point pattern (e.g., corresponding to a different fit for the patient's left and right breast respectively).

Furthermore, after shifting the entire sampling point pattern relative to the image mask, the system can individually shift sampling points relative to the image mask by sampling a sensor position distribution (typically a distribution with a lower variance) representing the variation in each temperature sensor in the elastic mesh relative to the whole mesh.

Both the fitting distribution and the sensor position distribution can be modeled according to actual observed variation in the fit and sensor positions in the temperature-sensing brassiere.

In one implementation, the system can provide an interface for a user to define a set of reference points that correspond to the same breast locations in the frontal and lateral images. These points include the nipples and a point in the upper and lower limits of the breast (edges of the breast mask, ensuring that the same breast location is marked in the frontal and lateral images). The system can then establish sampling point locations based on the reference points. In another implementation, the system can provide an interface to enable the user to define all sampling points manually in both the lateral and frontal thermographic images.

However, the sampling point pattern for a thermographic image mask of a patient's breasts can be defined in any other way.

### 1.7 Temperature Map Extraction

Once the system has distributed a sampling point pattern over the image mask, the system can then extract a temperature map from the thermographic image in Block S140. More specifically, for each sampling point in the set of sampling points (i.e. the sampling point pattern) the system can extract a temperature data point from the set of thermographic images to generate a temperature map in Block S140. For each sampling point, the system can reference the pixel in the image mask of the thermographic image in order to determine the color/hue of the pixel. The system can then convert the color/hue of the pixel into a temperature value according to a color-temperature correspondence key. The system can then store the temperature map for further processing and conversion into an input vector for the breast cancer screening model. Alternatively, the system can reference a matrix of temperatures values corresponding to each pixel in the thermographic image and extract temperature values corresponding to the set of sampling points in the sampling point pattern.

In one implementation, the system calculates a temperature data point corresponding to a sampling point by calculating a summary statistic of temperature value of the sampling point pixel and the temperature values of the surrounding pixels in order to reduce noise in the extracted temperature data points and to better approximate the sampling process of physical temperature sensors.

### 1.8 Screening Model

The system executes a breast cancer screening model that automatically calculates a breast cancer risk assessment, pathological classification, and/or identification of any breast abnormalities for the patient for the patient based on the temperature data from the temperature-sensing brassiere or from the aforementioned extraction process. The breast cancer diagnostic method can include a neural network or any other statistical method for classification of the temperature data. Furthermore, the breast cancer screening model can include an ensemble of statistical methods that together form a consensus output for each input vector derived from a temperature map.

In one implementation, the system can execute the breast cancer screening model to generate a report indicating a breast cancer risk assessment and statistical features that contributed to the breast cancer risk assessment. For example, the system can generate a report including a thermal map for each breast. The thermal map can indicate regions of the patient's breasts that most influenced the risk assessment. In another example, the report can include estimates of tumor parameters such as location, size, depth, and/or doubling time.

### 1.8.1 Input Vector

In order to train the breast cancer screening model for a temperature-sensing brassiere using temperature data extracted from thermographic images, the system can convert the temperature map extracted from the thermographic image into an input vector for the breast cancer screening model. More specifically, in Block S150, the system can generate an input vector based on the set of temperature data by normalizing, scaling, or calculating secondary data based on the temperature map.

In one implementation, the system organizes the temperature map as a one-dimensional array containing each data point in the temperature map. In this implementation, each location in the array corresponds to a particular sensor location in the temperature-sensing brassiere. Additionally or alternatively, the system can organize the temperature map as a two-dimensional array approximating (e.g., as a grid) the relative positions of the sampling point from which each temperature data point was extracted.

In another implementation, the temperature map stores each temperature data point in association with a pixel coordinate or location of the temperature data points relative to the other temperature data points in the temperature map. The system can then perform two-dimensional temperature interpolation between each temperature data point extracted from the image mask in order to produce a two-dimensional image as an input for the breast cancer screening model. Alternatively, the system can: generate a three-dimensional representation of the patient's breasts based on previously identified anatomical characteristics of the patient's breasts; project the temperature data point to a location on the three-dimensional model; interpolate over the surface of the three-dimensional model; and input the temperature interpolated three-dimensional model as the input vector for the breast cancer screening model.

Furthermore, the input vector for the breast cancer screening model can include other (non-temperature) data such as the patient's family history of cancer, age, ethnicity, or any other breast cancer risk factors of the patient.

However, the system can generate an input vector based on a temperature map, and any other data about the patient, in any other way such that the temperature data is included as a training example for the breast cancer screening model.

### 1.8.2 Training

Once the system has generated an input vector based on the temperature map, the system can input the input vector and the pathology label as a training example in a breast cancer screening model in Block S160. More specifically, the system can perform a training algorithm for the breast cancer screening model by: inputting the input vector into the breast cancer screening model; calculating a pathology output of the breast cancer screening model; comparing the pathology output of the breast cancer screening model with the pathology label associated with the input vector; and updating the breast cancer screening model based on the comparison. Thus, the system can implement any supervised learning algorithm appropriate for the breast cancer screening model. For example, the system can implement the breast cancer screening model as an artificial neural network and perform a backpropagation algorithm to train the artificial neural network. The system can implement one or more models/training algorithms such as support vector machines, linear regression logistic regression, naive Bayes, linear discriminant analysis decision trees, k-nearest neighbor algorithms, neural networks (e.g., pre-trained image recognition networks).

Furthermore, the system can execute unsupervised learning methods to achieve dimensionality reduction in the input vectors for the breast cancer screening model. Thus, the system can execute any unsupervised learning methods to enhance the breast cancer screening model.

### 2. Second Method

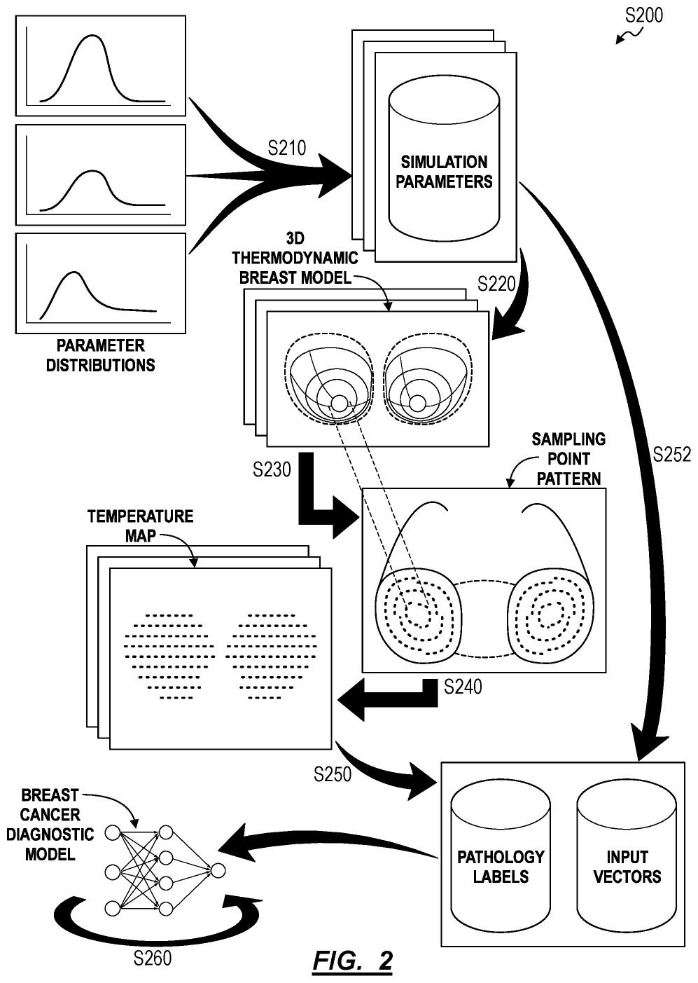

A shown in FIG. 2, a second method S200 for training a breast cancer screening model includes: obtaining a set of simulation parameters in Block S210; generating a thermodynamic simulation of a pair of breasts shaped according to the set of parameters and representing a simulated tumor within the pair of breasts located according to the set of simulation parameters in Block S220; distributing a set of sampling points over a surface of the thermodynamic simulation, the set of sampling points approximating locations of temperature sensors in a sensor mesh of a temperature sensing brassiere worn over the pair of breasts in Block S230; for each sampling point in the set of sampling points extracting a temperature data point from the thermodynamic simulation to generate a set of temperature data in Block S240; generating an input vector based on the set of temperature data in Block S250; generating a pathology label based on the set of tumor parameters and the set of tumor positions in Block S252; and inputting the input vector and the pathology label as a training example in a breast cancer screening model in Block S260.

### 2.1 Second Method: Applications

Generally, the second method S200 is executed by the system. The system executes the second method S200 in order to leverage three-dimensional thermodynamic simulations of human breasts to train a breast cancer screening model for a contact thermography device (e.g., the temperature-sensing brassiere). As previously described with reference to the first method S100, the accuracy of the breast cancer screening model may depend on the quantity and quality of the provided training data, which may take years to collect by administering tests with the temperature-sensing brassiere. However, the training period for the breast cancer screening model can be reduced by utilizing anatomically accurate simulated breast tissue to virtually obtain temperature data. Thus, the method S200 can simulate the temperature of virtual breasts (e.g., both healthy and abnormal breasts) and convert the external temperature of the virtual breasts into an input vector that can function as a training example for the breast cancer screening model.

In order to simulate a set of virtual breasts, the system accesses various parameter distributions that define the anatomy and/or pathology of the virtual breasts. For example, the system can access: a set of morphology distributions that define the shape, size, and position on the torso of the virtual breasts; a set of tumor parameter distributions that indicate the shape, size, and properties of a tumor; and a tumor position distribution that indicates the position of the virtual tumor within the virtual breasts.

The system can then generate a thermodynamic simulation of a three-dimensional model (e.g., a CAD model) of the virtual breasts and tumor(s) approximating anatomically accurate breasts. For example, the system can generate a model that includes glandular, adipose, muscular, and cutaneous regions of the virtual breasts. Additionally, the system can generate pathologically accurate models of tumors positioned within the virtual breasts. After generating the three-dimensional structure of the virtual breasts and tumors, the system can implement a finite element analysis (hereinafter “FEA”), a finite difference method (hereinafter “FDM”), a method of lines (hereinafter “MOL”), or any other method to solve the thermodynamic equations at each point in the virtual breasts and therefore the steady state and transient surface temperature distribution of the virtual breasts.

In an alternative implementation, the system can access a three-dimensional scan of a patient with corresponding thermographic data corresponding to points on the surface of the three-dimensional scan. In this implementation, the system can extract discrete temperature data points from the three-dimensional scan and thermographic surface data corresponding without performing a simulation of virtual breasts

After the system has generated a simulation of virtual breasts or accessed a three-dimensional thermographic scan of a patient, the system can distribute (e.g., automatically or via user input to an interface generated by the system), over the simulated virtual breasts, a set of sampling points that approximate the locations of temperature sensors in a temperature-sensing brassiere if worn over the virtual breasts. In order to distribute the sampling points in a pattern that approximates the distribution of temperature sensors of a temperature-sensing brassiere worn over the virtual breasts, the system can position the distribution of sampling points based on the locations of the virtual areolas of the virtual breasts. Alternatively, the system can position sampling points over the three-dimensional virtual breasts according to other reference geometry. Furthermore, the system can sample from a sampling point pattern in order to randomly offset each sampling point relative to the original distribution in order to represent random variation in the relative position of temperature sensors in the temperature-sensing brassiere.

Once the sampling points have been calculated, the system extracts temperature data from the thermodynamic simulation according to the calculated temperature value at each sampling point on the three-dimensional surface of thermodynamic simulation (or a summary statistic of the temperature data surrounding the sampling point in the thermodynamic simulation). Upon extracting the temperature data from the thermodynamic simulation, the system can store the extracted temperature data as an input vector for a breast cancer screening model. Additionally, the system can also perform further processing, such as data normalization, data interpolation, and/or data comparison between temperature data derived from the virtual breasts to generate the input vector for the breast cancer screening model.

The system can then pair each input vector with a corresponding pathology label describing the pathology of the patient. The system can determine the pathology label according to the virtual tumor(s) simulated in the thermodynamic simulation. For example, if the thermodynamic simulation included a tumor three centimeters in diameter, then the system can include the input vector and the pathology label in a supervised learning algorithm to train the breast cancer screening model. Alternatively, the system can include a full set of tumor parameters describing the tumors present in the virtual breasts in the training example for the breast cancer screening model.

For ease of explanation, the method S200 is described in the context of a temperature-sensing brassiere. However, the method S200 can be adapted for any other contact thermography device by modifying the sampling point pattern that corresponds to the sensor mesh of the temperature-sensing brassiere to a sampling point pattern that corresponds to an array of temperature sensors as configured on the contact thermography device. The system can also include other properties of the contact thermography device (e.g., the general structure, materials, and/or other physical properties of the device) in approximating the sampling point pattern for the thermographic image.

### 2.2 Thermodynamic Simulation

As shown in FIG. 2, the system can execute a thermodynamic simulation that can generate an accurate temperature profile for virtual breasts defined by the aforementioned simulation parameters in Block S220. More specifically, the system can generate a three-dimensional model of virtual breasts based on a set of sampled morphology parameters; and simulate the presence of a virtual tumor within the virtual breast based on a set of tumor parameters and the tumor position.

The system can sample a set of parameter distributions that provide a realistic (e.g., representative of a target patient population) set of parameter values for the thermodynamic simulation. More specifically, the system can: sample a set of morphology parameter distributions to obtain a set of morphology; sample a set of tumor parameter distributions to obtain a set of tumor parameters; and sample a tumor position distribution to obtain a set of tumor positions.

The set of morphology parameters can include breast diameter, breast length, breast volume, breast orientation, breast positioning on the torso, volume glandular fraction, areal glandular fraction, fibroglandular tissue distribution, breast density, and any other anatomical feature that can define breast shape or composition. The parameter distributions for each morphology parameter can be representative of a particular patient population or the population at large.

The set of tumor parameters can include tumor diameter, tumor density, and tumor perfusion rate, metabolic heat generation rate, or any characteristics of the tumor. The tumor parameter distributions can be determined based on tumor pathologies observed in a patient population or extrapolate trends in tumor size beyond typical detectable sizes. The tumor position is a separate parameter that indicates the relative location of the tumor within the virtual breast. The tumor location is a location within the volume of the virtual breast sampled from the tumor location distribution. In one implementation, the tumor location distribution is representative of observed tumor pathology in a patient population. Furthermore, the three-dimensional model can also sample parameters such as the number of tumors or parameters for benign masses, the thermal conductivity of the tumor, the specific heat of the tumor, the blood perfusion rate of the tumor, and the metabolic heat generation rate of the tumor.

The system can generate a three-dimensional model of virtual breasts, which can include multiple types of breast tissue (e.g. adipose, glandular, muscular, and skin tissue) arranged in an anatomically accurate shape (e.g., an anatomically accurate three-dimensional model). The system can perform the thermodynamic simulation to determine the surface temperature profile of the virtual breast based on the specific heat of each type of tissue in the model, the initial internal temperature of each tissue in the model, the arterial blood temperature, the blood perfusion rate for each tissue in the model, and the heat generation rate for each tissue in the model.

In one implementation, the thermodynamic model can include breast parts of differing tissue compositions, wherein the breast parts include the epidermis of the skin, the papillary dermis of the skin, the reticular dermis of the skin, adipose tissue, glandular tissue, and tumor tissue. For each of the breast parts, the model includes parameters such as the geometry of each part (thickness, radius, and/or location in the case of the one or more tumors), the density of each part, the Neo-Hookean range of each part, the elastic modulus of each part, the thermal conductivity of each part, the specific heat of each part, the blood perfusion rate of each part, and the heat generation rate of each part. The thermodynamic model can also include general parameters such as the convection coefficient of the surface of the skin, the temperature of the chest wall, the arterial blood temperature, and the ambient temperature.

In one implementation, the thermodynamic model can include performing FEA, FDM, MOL, or another numerical simulation technique to calculate the temperature profile at the surface of the skin based on the parameters of the model. The thermodynamic model can utilize the Pennes bio-heat equation to describe the propagation of heat through the various breast tissues (represented as finite elements). The Pennes bio-heat equation is described as follows:

$$ρ c \frac{\partial T}{\partial t} = ∇ ( k \cdot ∇ T ) + {W}_{b} \cdot {C}_{b} \cdot {ρ}_{b} ( {T}_{b} - T ) + {q}_{m}$$

where ρ is the density of the tissue, c is the heat capacity of the tissue, k is the thermal conductivity of the tissue, qₘ is the heat generation rate of the tumor, W_{b} is the blood perfusion rate, C_{b} is the blood heat capacity, ρ_{b} is the blood density, and T_{b} is the blood temperature.

In one implementation, the method S100 includes executing a GAN to implement the thermodynamic model. In this implementation, the generative network of the GAN generates the internal breast properties that best fit the temperature data and/or IMU data recorded by the temperature-sensing brassiere. The discriminative network evaluates whether the internal breast properties fit within a dataset of real internal breast properties. Alternatively, the system can execute analytic solutions to the Pennes bio-heat equation, evolutionary algorithms, or other analytical methods to simulate the temperature distribute of the virtual breasts.

However, the method S100 can include executing any other machine learning model in order to implement the thermodynamic model.

### 2.3 Three-Dimensional Thermographic Scan

In one implementation, the breast morphology is not generated, but rather taken from a three-dimensional scan of patient. Thus, instead of performing a simulation of virtual breasts from parameters alone, the system can access a three-dimensional scan; adjust a template three-dimensional mesh to the three-dimensional thermographic scan; and generate a thermodynamic virtual breast simulation based on the three-dimensional scan. In order to generate a thermodynamic virtual breast simulation, the system can interpolate an artificial mesh of the three-dimensional scan to generate a continuum of viable artificial meshes.

However, the system can transform three-dimensional scan data into an anatomically accurate thermodynamic model of virtual breasts in any other way.

### 2.4 Sampling Point Pattern

The system can distribute a set of sampling points over a surface of the thermodynamic simulation and/or a three-dimensional thermographic model, the set of sampling points approximating locations of temperature sensors in a sensor mesh of a temperature sensing brassiere worn over the pair of breasts in Block S230. Similar to the sampling point pattern for the thermographic images described with reference to Block S130, the system can estimate a sampling point pattern that approximates the sensor locations of temperature sensors in a temperature-sensing brassiere worn over the pair of virtual breasts. However, in this case, the system can project estimated sensor locations directly onto the three-dimensional thermodynamic model of the virtual breasts based on the estimated sensor locations for the pair of virtual breasts. The system can then sample from the predetermined set of sampling points to obtain a set of temperature data for the set of virtual breasts that is equivalent to a temperature map obtained from a thermographic image or from the temperature-sensing brassiere itself in Block S240. Thus, the system can perform the methods described above with respect to the first method S100 to generate an input vector based on the temperature data in Block S250 (equivalent to Block S140), and train the breast cancer screening model in Block S260 (equivalent to Block S150).

### 2.5 Pathology Label

The system can also generate a pathology label based on the set of tumor parameters and the set of tumor positions in Block S252. The system can use a decision tree or logical programming to select an appropriate pathology or risk factor that medically corresponds to the sampled tumor distributions and tumor locations within the virtual breast.

### 3. Combination of the First Method and the Second Method

In one variation, the system can execute the first method S100 and the second method S200 in order to train a single breast cancer screening model. In this variation, the system executes the first method S100 and the second method S200 consecutively and in parallel and performs the training steps (S160 and S260) using training examples generated via executing both the first method S100 and the second method S200.

### 4. Temperature Sensing Brassiere

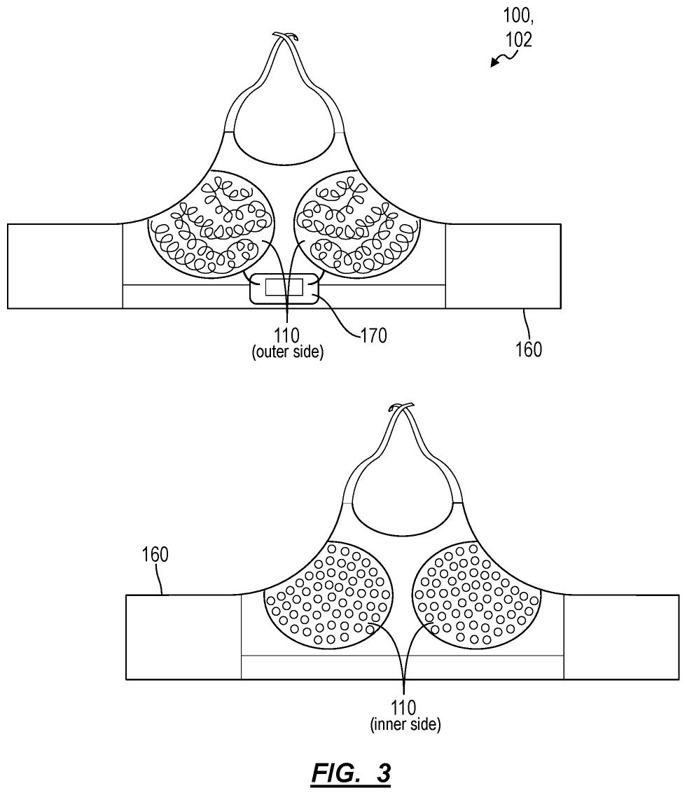

Generally, as shown in FIG. 3, the temperature-sensing brassiere 102 includes a set of temperature sensors 134 that are evenly distributed across surfaces of a user's breasts in a manner that facilitates normal circulation (e.g., similar to circulation in the user's breasts when the user is not wearing the temperature-sensing brassiere 102), thereby enabling collection of spatially consistent temperature data across multiple users characterized by variable breast sizes. In particular, the temperature-sensing brassiere 102 can include: a pair of sensor meshes 110 (one for each breast of the user) for distributing a set of temperature sensors 134 across each breast of the user; and a brassiere structure that locates each sensor mesh 110 over each breast of the user. The temperature-sensing brassiere 102 can further include: one or more IMUs arranged within the brassiere structure in order to detect changes in the user's posture during the data collection period and/or to detect the size and/or shape of the user's breasts; a controller 150, arranged within the brassier structure in order to sample data from the set of temperature sensors 134 in the temperature-sensing brassiere 102, and any IMUs of the temperature-sensing brassiere 102; and a battery arranged within the brassiere structure in order to provide electrical power to the controller 150.

More specifically, the temperature-sensing brassiere 102 can include a pair of sensor meshes 110 defining a mesh pattern (e.g., a triangle mesh, a quadrilateral mesh, a hybrid mesh) and each including a flexible mesh 120 structure (i.e. a flexible mesh 120) and a set of temperature-sensing assemblies 130. Thus, the temperature-sensing brassiere 102 can include: a first flexible mesh 120 extending from immediately right of a sternum of the user across a right breast of the user to a right axillary region of the user and from below the right breast of the user across the right breast of the user to a right clavicular region of the user, when the temperature-sensing brassiere 102 is worn by the user; and a second flexible mesh 120 extending from immediately left of a sternum of the user across a left breast of the user to a left axillary region of the user and from below the left breast of the user across the left breast of the user to a left clavicular region of the user, when the temperature-sensing brassiere 102 is worn by the user. Each of the first sensor mesh 110 and the second sensor mesh 110 can further include a set of temperature sensing assemblies arranged at intersections in the sensor meshes 110 (i.e. a first set of temperature-sensing assemblies 130 arranged at intersections in the first flexible mesh 120 and a second set of temperature-sensing assemblies 130 arranged at intersections in the second flexible mesh 120).

Additionally, the temperature-sensing brassiere 102 includes an electrical connection (i.e. an electrical trace 140 or wire) for each of the temperature-sensing assemblies 130 that electrically couples each temperature-sensing assembly 130 to the controller 150 via the flexible mesh 120, thereby enabling the controller 150 to sample a temperature sensor 134 in each temperature-sensing assembly 130 during the data collection period.

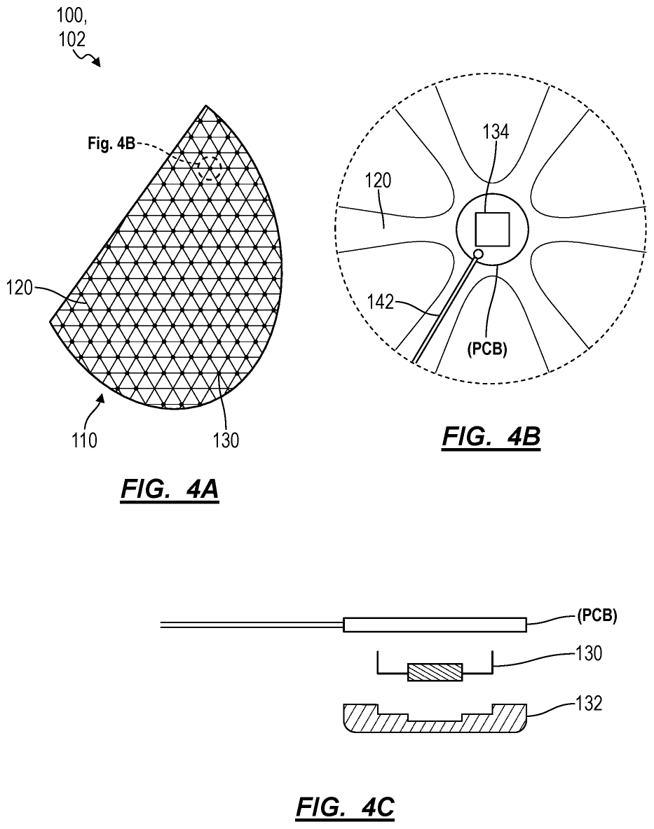

As shown in FIGS. 4A, 4B, and 4C, in another variation of the temperature-sensing brassiere 102, the flexible mesh 120 is a fabric mesh supporting temperature-sensing assemblies 130 at each intersection of the fabric mesh. This variation additionally includes elastic wires 142 arranged along the fabric mesh electrically coupling each temperature-sensing assembly 130 to the controller 150.

The temperature-sensing brassiere 102 can be secured to the user in a similar manner to a typical brassier. However, instead of a typical cup, the temperature-sensing brassiere 102 includes a pair of sensor meshes no for positioning a set of temperature sensors 134 across each of the user's breasts (i.e. a left mesh and a right mesh, or a single mesh for a post-mastectomy user). The temperature-sensing brassiere 102 can also include a power source, a controller 150, wireless chip (e.g. a Wifi or Bluetooth chip), and/or a wired port as means for recording, formatting, and/or transmitting data received from the temperature sensors 134 in the left sensor mesh 110 and the right sensor mesh 110 to another computational device 106 to perform post-processing and analysis of the temperature data.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for training a breast cancer screening model comprises:
accessing a set of thermographic images of a torso of a patient, the set of thermographic images associated with a pathology label; and
for each of the set of thermographic images:
calculating an image mask defining a breast area in the thermographic image;
within the image mask, distributing a set of sampling points of each thermographic image, the set of sampling points approximating locations of temperature sensors in a sensor mesh of a temperature sensing brassiere if the temperature sensing brassiere were worn over the torso of the patient;
for each sampling point in the set of sampling points, extracting a temperature data point from the set of thermographic images to generate a temperature map;
generating an input vector based on the temperature map; and
inputting the input vector and the pathology label as a training example in a breast cancer screening model.
